SEQUENCE LISTING (4 sequences)

SEQ ID NO: 1 - DNA, 231 bp, Lactobacillus reuteri
  misc_feature 1..231: note = Description of sequence: mersacidin (mersacidin-E1)
  source 1..231: mol_type = genomic DNA
atggacaaagaagaattagaaaaaattgtaggtaataactttgaggaaatgagtttacaaaaaatgacagaaattcaaggtatgggtgaataccaagtggattcaacaccagcagcttctgcgatttcacgggcaacaattcaagtatcacgtgcatcttctggaaaatgtctaagttggggtagtggtgcagcatttagtgcttattttactcataaaagatggtgctag

SEQ ID NO: 2 - protein, 76 aa, Lactobacillus reuteri
  REGION 1..76: note = Description of sequence: mersacidin (mersacidin-E1)
MDKEELEKIVGNNFEEMSLQKMTEIQGMGEYQVDSTPAASAISRATIQVSRASSGKCLSWGSGAAFSAYFTHKRWC

SEQ ID NO: 3 - DNA, 186 bp, Lactobacillus reuteri
  misc_feature 1..186: note = Description of sequence: mersacidin (mersacidin-E2)
  source 1..186: mol_type = genomic DNA
atggaagaaaaagaattagaaggtgtaatagggaattcgtttgaaagtatgactgtagaggaaatgacaaaaattcaaggtatgggtgaatatcaagtagattcgacgcctggatattttatggaaagtgctgccttttcagctcttacagccaatataacaagacatgctatgcatcatcattaa

SEQ ID NO: 4 - protein, 61 aa, Lactobacillus reuteri
  REGION 1..61: note = Description of sequence: mersacidin (mersacidin-E2)
MEEKELEGVIGNSFESMTVEEMTKIQGMGEYQVDSTPGYFMESAAFSALTANITRHAMHHH

(19) United States
(12) Patent Application Publication
(10) Pub. No.: US 2024/0390431 A1
(43) Pub. Date: Nov. 28, 2024

(54) Methods of Inhibiting Diseases Caused by Respiratory Viruses

(71) Applicant: BiomEdit, LLC, Fishers, IN (US)

(72) Inventors: Arvind Kumar, Fishers, IN (US); Dharanesh Mahimapura Gangaiah, Fishers, IN (US)

(73) Assignee: BiomEdit, LLC, Fishers, IN (US)

(21) Appl. No.: 18/694,118

(22) PCT Filed: Sep. 22, 2022
(86) PCT No.: PCT/US2022/044454

§ 371 Date: Mar. 21, 2024

Related U.S. Application Data
(60) Provisional application No. 63/247,275, filed on Sep. 22, 2021.

(51) Int. Cl. A61K 35/741; A61K 35/00

(52) U.S. Cl. CPC A61K 35/741; A61K 2035/115

(57) ABSTRACT

The present invention provides a method of inhibiting a viral respiratory disease, comprising: administering to a subject, in need thereof, an effective amount of L. reuteri strain 3632 and/or strain 3630, wherein L. reuteri strain 3632 and/or strain 3630 secrete mersacidin.

### BACKGROUND OF THE INVENTION

Coronaviruses (CoV) are a large family of viruses that cause illnesses ranging from the common cold to more severe diseases such as Middle East Respiratory Syndrome (MERS), Severe Acute Respiratory Syndrome (SARS) and Coronavirus disease 2019 (CoViD-19). Coronaviruses are zoonotic, meaning they are transmitted between animals and people, e.g., SARS-COV from civet cats to humans; MERS-COV from dromedary camels to humans. Several known coronaviruses are circulating in animals that have not yet infected humans.

Coronavirus Disease 2019 (CoViD-19) is an infectious disease caused by the Severe Acute Respiratory Syndrome-Coronavirus-2 (SARS-COV-2). This disease has spread globally since 2019, resulting in the 2019-2021 coronavirus pandemic.

Common symptoms of CoViD-19 include fever, cough, shortness of breath, and loss of taste/smell sensations. Muscle pain, sputum production and sore throat are some of the less common symptoms. While the majority of cases result in mild symptoms, some progress to pneumonia, severe acute respiratory syndrome, multi-organ failure and even death. The case fatality rate is estimated at between 1% and 5% but varies by age and other health conditions. The disease is more likely to occur in people whose immune system is impaired due to age, immunosuppressive therapy, psychological stress, or other factors.

The infection is spread from one person to others via respiratory droplets, often produced during coughing. Time from exposure to onset of symptoms is generally between two and fourteen days, with an average of five days. The standard method of diagnosis is by reverse transcription polymerase chain reaction (rRT-PCR) from a nasopharyngeal swab or sputum sample. Antibody assays are performed using a blood serum sample. The infection can also be diagnosed from a combination of symptoms, risk factors and a chest CT scan showing features of pneumonia.

Recommended measures to prevent the transmission of CoViD-19 include frequent hand washing, maintaining distance from other people, not touching one's face, covering mouth/nose when coughing/sneezing, and the use of masks. CoViD-19 is a poorly understood disease with unique clinical/transmission characteristics, evinced by global health experts who provide daily updates with varying medical advice.

Accordingly, an urgent need exists for an effective and safe method of preventing and/or inhibiting CoViD-19, and the symptoms thereof.

### SUMMARY OF THE INVENTION

The present invention relates to methods of treating viral respiratory diseases and associated diseases. In one aspect, the method comprises administering to a subject, in need thereof, an effective amount of L. reuteri strain 3632 and/or strain 3630. An effective amount is an amount wherein L. reuteri strain 3632 and/or strain 3630 secrete an effective amount of mersacidin. In one embodiment, L. reuteri strain 3632 and/or strain 363 is administered by probiotic composition. In one embodiment, L. reuteri strain 3632 and/or strain 363 is administered by a live delivery platform. In one embodiment, the administration is oral, by inhalation, intravenous, subcutaneous, intramuscular or mucosal. In one embodiment, the disease is caused by a corona virus or influenza virus. In one embodiment, the corona virus is selected from the group consisting of alpha coronaviruses, the beta coronaviruses, MERS-COV, SAR-COV, SARS-COV-2 and the common cold.

In one aspect, the present invention relates to methods of treating a viral respiratory disease comprising: systemically administering to a subject, in need thereof, a pharmaceutical composition comprising an effective amount of mersacidin. In one embodiment, the administration is oral, by inhalation, intravenous, subcutaneous, intramuscular or mucosal. In one embodiment, the disease is caused by a corona virus or influenza virus.

In one aspect, the present invention provides methods of treating gut dysbiosis comprising: administering to a subject, in need thereof, an effective amount of L. reuteri strain 3632 and/or strain 3630, wherein L. reuteri strain 3632 and/or strain 3630 secrete mersacidin. In one embodiment, L. reuteri strain 3632 and/or strain 363 is administered by probiotic composition. In one embodiment, L. reuteri strain 3632 and/or strain 363 is administered by a live delivery platform.

In one aspect, the present methods include treating gut dysbiosis comprising: systemically administering to a subject, in need thereof, a pharmaceutical composition comprising an effective amount of mersacidin. In one embodiment, the administration is oral, by inhalation, intravenous, subcutaneous, intramuscular or mucosal.

### BRIEF DESCRIPTION OF THE DRAWINGS

FIG. 1A shows Lr 3632 expressed Lanthipeptide Mersacidin 1 potentially binds and interferes with CoViD-19 spike protein interaction with ACE2 receptor.

FIG. 1B shows that given only 1-3 mutations at most at the Receptor Binding Domain (RBD) for the variants WT spike, P.1 spike and B.1.351 spike, they are not large enough to considerably disrupt mersacidin binding.

FIG. 2 shows Elanco® probiotic strains administered along with a coronavirus vaccine in a poultry study improved outcomes across two key metrics. The strains also had a material standalone impact on improving immunity across all four metrics measured.

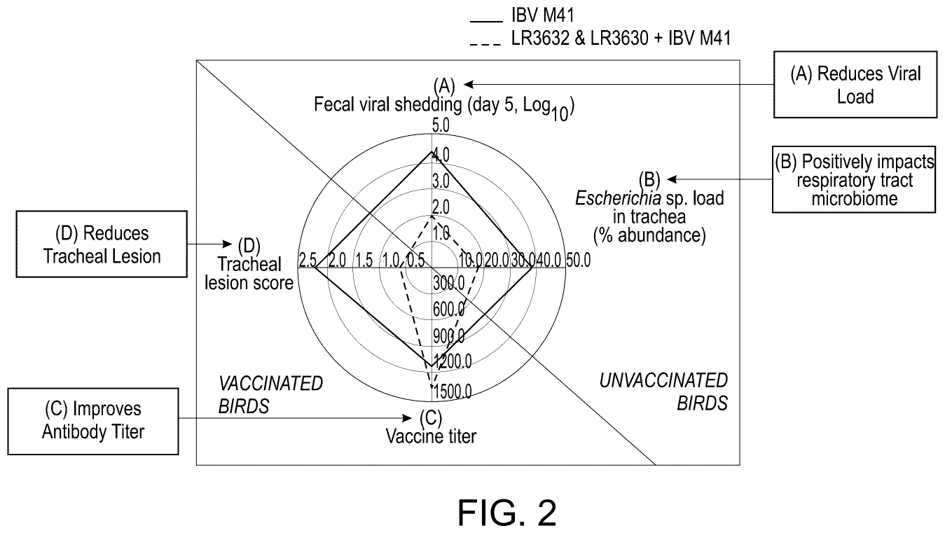

FIG. 3 shows a homology model of mersacidin 1.

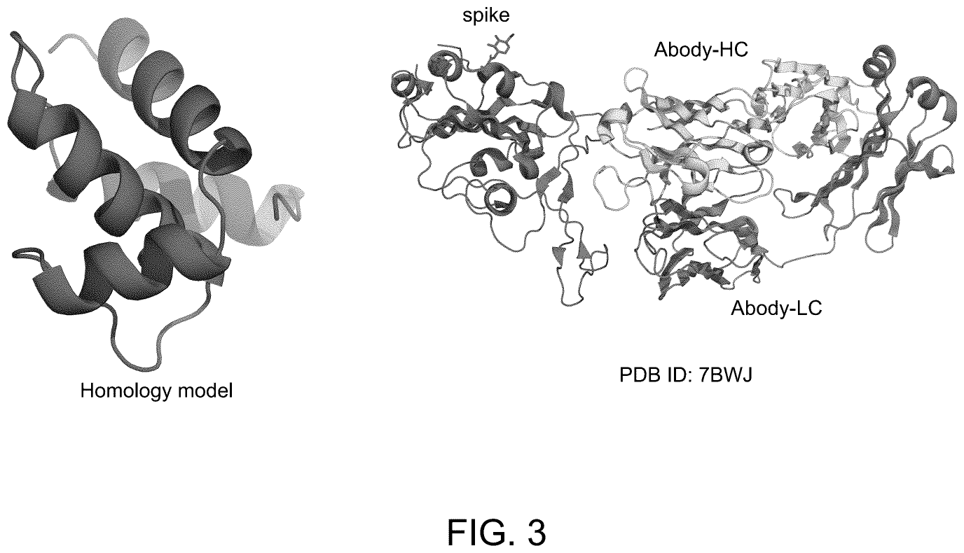

FIG. 4 shows that with alignment of multiple decoys, mersacidin 1 prefers binding to RBD. Red is mersacidin 1; Blue is Spike Protein and RBD.

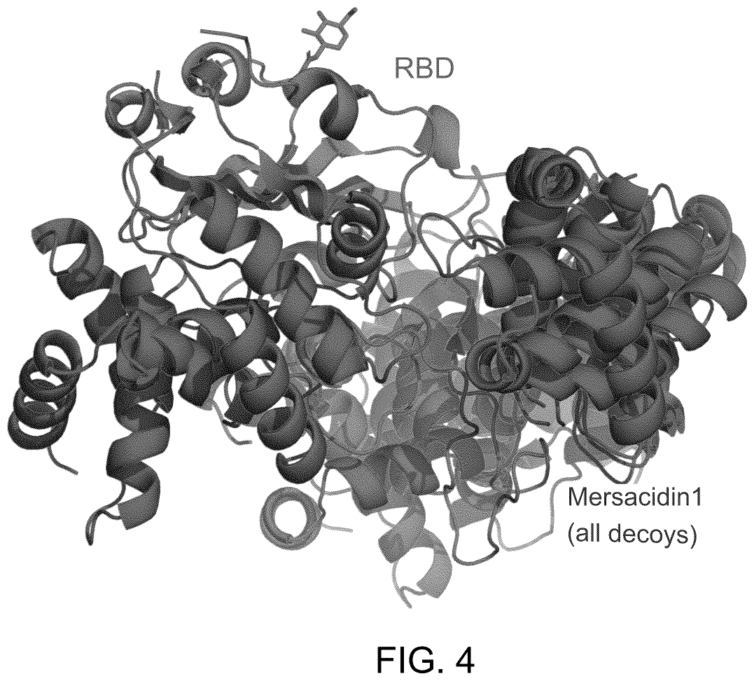

FIG. 5 shows a comparison of binding modes of mersacidin 1 (top candidate) vs. ACE2.

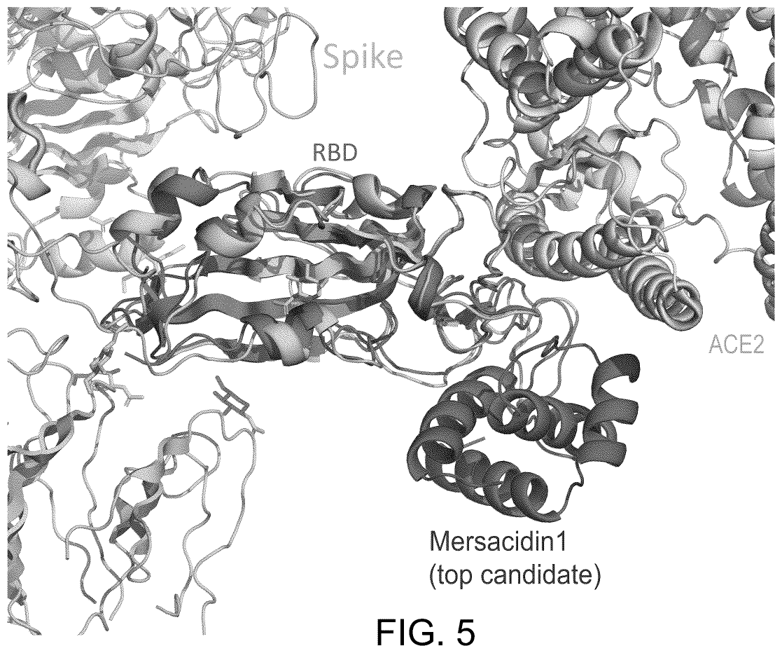

FIG. 6 shows a comparison of binding modes of mersacidin 1 (all possible decoys) vs. ACE2.

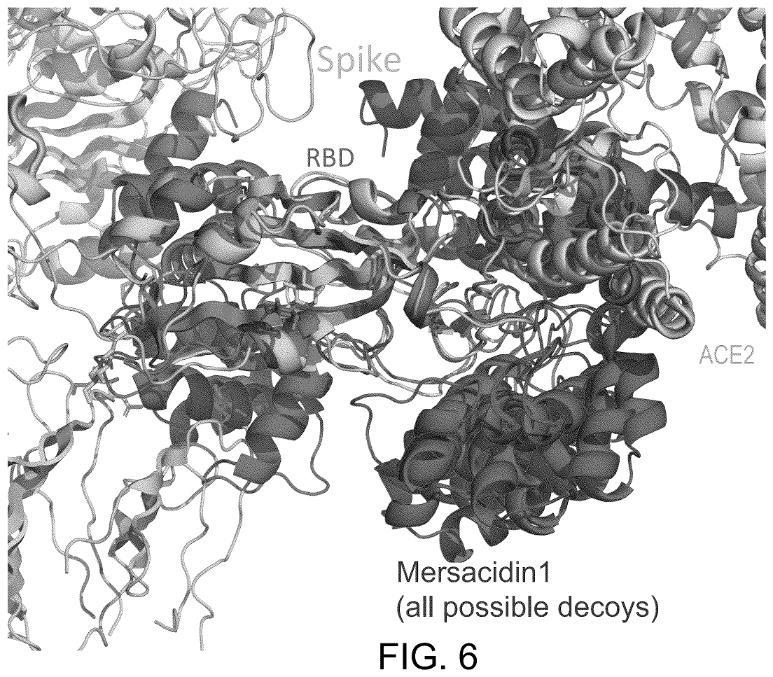

### DETAILED DESCRIPTION OF THE INVENTION

In one aspect, the methods of the present invention provide treatments and inhibition of respiratory diseases. The methods comprise the direct or indirect systemic administration of mersacidin to a human or animal in an amount which is effective for the treatment and/or inhibition of respiratory diseases, and the symptoms thereof. Examples of respiratory diseases are diseases caused by corona viruses and influenza viruses.

Examples of corona viruses for which the present invention is suitable include SARS-COV-2, SAR-COV, MERS-COV, the alpha coronaviruses (e.g., 229E, NL63), and the beta coronaviruses (e.g., OC43, HKU1). SARS-COV-2 causes Coronavirus Disease 2019 (i.e., CoViD-19, and its variants (e.g., delta)). SARS-COV causes Severe Acute Respiratory Syndrome (i.e., SARS). MERS-COV causes Middle East Respiratory Syndrome (i.e., MERS). The alpha and beta coronaviruses usually cause mild to moderate upper-respiratory tract illnesses, like the common cold.

Examples of Influenza Viruses for which the present invention is suitable include Swine Influenza Viruses (SIVs) and Avian Influenza Viruses. The known SIV strains include Influenza C and the subtypes of Influenza A known as H1N1, H1N2, H3N1, H3N2 and H2N3. Influenza A virus subtype H1N1 (A/H1N1) was the most common cause of human influenza in 2009, and is associated with the 1918 outbreak known as the Spanish flu. An emerging Avian Influenza Virus is the Highly Pathogenic Avian Influenza A virus subtype H5N1. Since the first human H5N1 outbreak in 1997, there has been an increasing number of HPAI H5N1 bird-to-human transmissions, leading to clinically severe and fatal human infections.

In one aspect, the methods of the present invention provide treatments and inhibition of gut dysbiosis. Gut dysbiosis causes severe intestinal lesions, compromised intestinal integrity (diarrhea in over 60% of patients), shed in feces and depletion of mucosal lymphoid cells. CoViD-19 is sometimes characterized by gut dysbiosis.

Animals which can benefit from the methods of the present invention include birds, humans, or non-human mammals. Examples of animals include pets (e.g., dogs, cats, etc.), livestock (e.g., cows, cattle, pigs, etc.), birds and laboratory animals (e.g., rodents, primates, etc.). Specific examples of birds include poultry such as chickens or turkey. Examples of chicken include broiler chickens or egg-laying or egg-producing chickens.

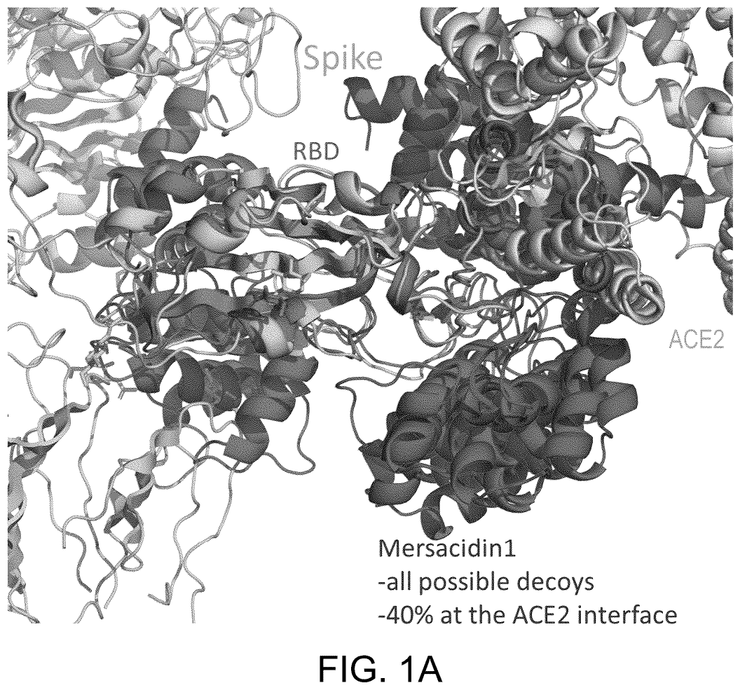

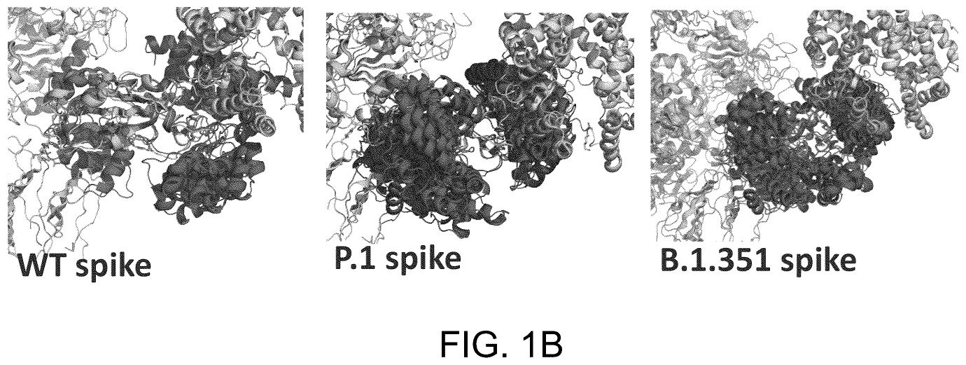

Mersacidin is an antimicrobial peptide, classified as a lantibiotic, containing beta-methyllanthionine. Administration of mersacidin inhibits and treats viral respiratory diseases (e.g., CoViD)). Without wanting to be limited to a mechanism of action, it is believed that mersacidin can bind the CoViD spike protein and/or interfere with its function, for example, by interfering with interaction between the ACE2 receptor and the CoViD spike protein. See FIG. 1A.

Mersacidin can be produced and secreted by a species of Bacillus. In particular, the Lactobacillus reuteri strain 3632 encodes for two bacteriocins belonging to mersacidin family based on homology to the mersacidin conserved domain. These bacteriocins appear to be unique to strain 3632. A cDNA encoding one mersacidin (mersacidin-E1) is:

| (SEQ ID NO: 1) | |
|---|---|
| 1 | atggacaaag aagaattaga aaaaattgta ggtaataact ttgaggaaat gagtttacaa |
| 61 | aaaatgacag aaattcaagg tatgggtgaa taccaagtgg attcaacacc agcagcttct |
| 121 | gcgatttcac gggcaacaat tcaagtatca cgtgcatctt ctggaaaatg tctaagttgg |
| 181 | ggtagtggtg cagcatttag tgcttatttt actcataaaa gatggtgcta g. |

This novel open reading frame would encode a polypeptide of mersacidin-E1:

(SEQ ID NO: 2)
MDKEELEKIVGNNFEEMSLQKMTEIQGMGEYQVDSTPAASAISRATIQV
SRASSGKCLSWGSGAAFSAYFTHKRWC.

Another cDNA encoding the second form of mersacidin (mersacidin-E2) is:

| (SEQ ID NO: 3) | |
|---|---|
| 1 | atggaagaaa aagaattaga aggtgtaata gggaattcgt ttgaaagtat gactgtagag |
| 61 | gaaatgacaa aaattcaagg tatgggtgaa tatcaagtag attcgacgcc tggatatttt |
| 121 | atggaaagtg ctgccttttc agctcttaca gccaatataa caagacatgc tatgcatcat |
| 181 | cattaa. |

This novel open reading frame would encode a polypeptide of mersacidin-E2:

(SEQ ID NO: 4)
MEEKELEGVIGNSFESMTVEEMTKIQGMGEYQVDSTPGYFMESAAFSAL
TANITRHAMHHH.

The Lactobacillus reuteri strain 3632 was deposited on 19 Jun. 2020 according to the Budapest Treaty in the ATCC Patent Depository and assigned ATCC Patent Deposit Number PTA-126788. Another Lactobacillus reuteri strain, strain 3630, was deposited on 19 Jun. 2020 in the ATCC Patent Depository and assigned ATCC Patent Deposit Number PTA-126787.

The L. reuteri strains 3630 and 3632 are described as probiotic strains in “Probiotic Compositions Comprising Lactobacillus Reuteri Strains and Methods of Use,” PCT/US2020/016668, filed Feb. 4, 2020, published as WO 2020/163398, Aug. 13, 2020, claiming priority to U.S. Ser. No. 62/801,307, filed Feb. 5, 2019; published as US 2022/0088094, Mar. 24, 2022, and US 2022/0125860, Apr. 28, 2022, all of which are incorporated herein by reference in their entireties.

In some embodiments, mersacidin is delivered to animals by direct fed microbials (DFMs), also called probiotic compositions. That is, Lactobacillus reuteri strains 3630 and 3632 are suitable as direct fed microbials, i.e., a probiotic composition is administered which contains bacteria that secrete mersacidin. Probiotic Compositions comprising L. reuteri strains 3630 and 3632 are described in “Immunogenic Probiotic Compositions,” PCT/US2021/058779, filed Nov. 10, 2021, published as WO 2022/103837, May 17, 2022, claiming priority to U.S. Ser. No. 63/111,979, filed Nov. 10, 2020, and US Provisional Patent Application 63/230,551, filed Aug. 6, 2020, which are incorporated herein by reference in their entireties.

The Lactobacillus reuteri strains 3630 and 3632 are also suitable for genetic modification and as live delivery or production strains of mersacidin. In some embodiments, the bacteria strains are delivered by being placed on a live delivery platform. For example, a live delivery system based on L. reuteri strain 3630 or 3632 is described in “A Genetically Modified Lactobacillus and Uses Thereof,” PCT/US2020/016522, filed Feb. 4, 2020, published as WO 2020/163284, Aug. 13, 2020, claiming priority to U.S. Ser. No. 62/801,307, filed Feb. 5, 2019, which is incorporated herein by reference in its entirety.

In some embodiments, the probiotic compositions and delivery systems comprise a combination of isolated Lactobacillus strains 3632 (PTA-126788) and 3630 (PTA-126787) or a lactobacillus strain having at least 98% or 99% amino acid or nucleic acid identity to such strains, while retaining capability to secrete mersacidin. In some embodiments, probiotic compositions and delivery systems include an isolated first L. reuteri strain and an isolated second L. reuteri strain at a ratio of about 0.75-1.5:1, more typically, about 1:1.

In some embodiments, the compositions comprise the isolated first L. reuteri strain and/or the isolated second L. reuteri strain in an amount of about 10²-10⁸ CFU/kg of the composition, about 10⁶-10⁸ CFU/kg of the composition, about 10⁴-10⁷ CFU/kg of the composition, about 10³-10⁵ CFU/kg of the composition, about 10² CFU/kg of the composition, about 10³ CFU/kg of the composition, about 10⁶ CFU/kg of the composition, about 10⁷ CFU/kg of the composition, or about 10⁸ CFU/kg of the composition. In some embodiments, the compositions comprise the isolated first L. reuteri strain in an amount of about 10²-10⁸ CFU/ml of the composition, about 10⁶-10⁸ CFU/ml of the composition, about 10⁴-10⁷ CFU/ml of the composition, about 10³-10⁵ CFU/ml of the composition, about 10³ CFU/ml of the composition, about 10⁴ CFU/ml of the composition, about 10⁵ CFU/ml of the composition, about 10⁶ CFU/ml of the composition, about 10⁷ CFU/ml of the composition, or about 10⁸ CFU/ml of the composition. Amounts will vary; routine experimentation will establish the required amount. Increasing amounts or multiple dosages may be implemented and used as needed.

In some embodiments, the probiotic compositions can be formulated as animal feed, feed additive, food ingredient, water additive, water-mixed additive, consumable solution, consumable spray additive, consumable solid, consumable gel, injection, or combinations thereof. In one embodiment, the composition includes water. For example, the strain(s) can be administered in the form of a supplement capsule, which strain(s) produces mersacidin in the digestive system.

As used herein, “delivery” or “administration” means the act of providing a beneficial activity to a host. The delivery may be direct or indirect. An administration could be by an oral, ocular, nasal, inhalation, parenteral, or mucosal route. Parental administration includes subcutaneous, intramuscular and intravenous administration. For example without limitation, an oral route may be an administration through drinking water, animal feed, or edible solid, a nasal route of administration may be through a spray or vapor, and a mucosal route of administration may be through direct contact with mucosal tissue (e.g., inside surface of the nose, mouth, esophagus, trachea, lungs, stomach, gut, intestines, and anus). In some embodiments, administration is by way of injection or infusion. Administration to an animal may be by any known or standard technique. These include oral ingestion, gastric intubation, or broncho-nasal spraying. In one embodiment, the composition is administered to a cow by way of intra-mammary infusion. The compositions disclosed herein may be administered by immersion, intranasal, intramammary, topical, mucosally, or inhalation. In the case of birds, administration may be in ovo, i.e. administration to a fertilized egg. In ovo administration can be via a liquid which is sprayed onto the egg shell surface, or an injected through the shell. Mucosal route of administration includes administration by inhalation of spray, aerosol, nebulized material, or vapor. When administering to animals, including farm animals, administration may include orally or by injection. Oral administration can include by bolus, tablet or paste, or as a powder or solution in feed or drinking water. The method of administration will often depend on the species being feed or administered, the numbers of animals being fed or administered, and other factors such as the handling facilitie available and the risk of stress for the animal.

In some embodiments, the method does not comprise administration of an antibiotic.

In some embodiments, the compositions and delivery systems additionally include one or more prebiotic. Prebiotics may include organic acids or non-digestible feed ingredients that are fermented in the lower gut and may serve to select for beneficial bacteria. Prebiotics may include mannan-oligosaccharides, fructo-oligosaccharides, galacto-oligosaccharides, chito-oligosaccharides, isomalto-oligosaccharides, pectic-oligosaccharides, xylo-oligosaccharides, and lactose-oligosaccharides.

In some embodiments, the compositions further include one or more components or additives. The component or additive can be a component or additive to facilitate administration, for example by way of a stabilizer or vehicle, or by way of an additive to enable administration to an animal such as by any suitable administrative means, including in aerosol or spray form, in water, in feed or in an injectable form.

The probiotic compositions and delivery systems may include a carrier in which the bacterium or any such other components is suspended or dissolved. Such carrier(s) may be any solvent or solid or encapsulated in a material that is non-toxic to the inoculated animal and compatible with the organism. Suitable pharmaceutical carriers include liquid carriers, such as normal saline and other non-toxic salts at or near physiological concentrations, and solid carriers, such as talc or sucrose and which can also be incorporated into feed for farm animals. When used for administering via the bronchial tubes, the composition is preferably presented in the form of an aerosol. A dye may be added to the compositions hereof, including to facilitate chacking or confirming whether an animal has ingested or breathed in the composition.

As used herein, “probiotic” refers to a substantially pure microbe (i.e., a single isolate) or a mixture of desired microbes, and may also include any additional components (e.g., carrier) that can be administered to an animal to provide a beneficial health effect. Probiotics or microbial compositions of the invention may be administered with an agent or carrier to allow the microbes to survive the environment of the gastrointestinal tract, i.e., to resist low pH and to grow in the gastrointestinal environment.

As used herein, “carrier”, “acceptable carrier”, or “pharmaceutical carrier” are used interchangeably and refer to a diluent, excipient, or vehicle with which the compound is administered. Such carriers can be sterile liquids, such as water and oils, including those of petroleum, animal, vegetable, or synthetic origin; such as peanut oil, soybean oil, mineral oil, sesame oil, and the like. Water or aqueous solution saline solutions and aqueous dextrose and glycerol solutions are preferably employed as carriers, in some embodiments as injectable solutions. Alternatively, the carrier can be a solid dosage form carrier, including but not limited to one or more of a binder (for compressed pills), a glidant, an encapsulating agent, a flavorant, and a colorant. The choice of carrier can be selected with regard to the intended route of administration and standard pharmaceutical practice. See Handbook of Pharmaceutical Excipients, (Sheskey, Cook, and Cable) 2017, 8th edition, Pharmaceutical Press; Remington's Pharmaceutical Sciences, (Remington and Gennaro) 1990, 18th edition, Mack Publishing Company; Development and Formulation of Veterinary Dosage Forms (Hardee and Baggot), 1998, 2nd edition, CRC Press.

As used herein, the terms “treating,” “to treat,” or “treatment” include restraining, slowing, stopping, inhibiting, reducing, ameliorating, or reversing the progression or severity of an existing symptom, disorder, condition, or disease. A treatment may also be applied prophylactically to prevent or reduce the incidence, occurrence, risk, or severity of a clinical symptom, disorder, condition, or disease.

In some embodiments, mersacidin itself can be directly administered as a pharmaceutical composition; the mersacidin being encoded by SEQ ID NO:1 or SEQ ID NO: 3, or by sequences with about at least 98% or 99% identity to SEQ ID NOs: 1 or 3; or the mersacidin having SEQ ID NO: 2 or SEQ ID NO: 4, or having at least about 98% or 99% identity to SEQ ID NO: 2 or SEQ ID NO:4. Methods of administering the mersacidin include, for example, orally, by inhalation, intravenously, subcutaneously and intramuscularly. In some embodiments, mersacidin is administered by buccal, intranasal and transdermal routes, as well as novel delivery systems such as the use of protective liposomes.

In one embodiment, mersacidin is in the form of physiologically tolerated salts and chemical equivalents. Physiologically tolerated salts of mersacidin can be formed in a generally known manner with a wide variety of compounds, for example with organic amines such as, for example, triethylamine or tri-(2-hydroxyethyl)-amine, with alkali metals and alkaline earth metals, such as sodium, potassium, magnesium and calcium, with inorganic acids such as, for example, hydrochloric acid, sulfuric acid or phosphoric acid and with organic acids such as, for example, acetic acid, citric acid, benzoic acid, maleic acid, fumaric acid, tartaric acid or p-toluene-sulfonic acid.

The actual preferred amounts of a pharmaceutical composition in a specific case will vary according to the particular compositions formulated, the mode of application, and the subject being treated (e.g., age, gender, size, tolerance to drug, etc.), as would be known to a skilled artisan with the foregoing guidance. Generally the amount of the mersacidin may be an amount sufficient to achieve dissolved concentrations of active compound on the airway surfaces of the subject of from about 10⁻⁹ to 10⁻³ moles/liter, and more preferably from 10⁻⁷ to 10⁻⁵ moles/liter. A daily dose may be divided among one or several unit dose administrations. Preferably, the daily dose is a single unit dose, which is preferably administered from 1 to 3 times a week. Treatments may continue week to week on a chronic basis as necessary (i.e., the active agent can be administered chronically). Administration of the active compounds may be carried out therapeutically (i.e., as a rescue treatment) or prophylactically.

Aerosols of liquid particles comprising mersacidin may be produced by any suitable means, such as with a nebulizer. Nebulizers are commercially available devices which transform solutions or suspensions of the active ingredient into a therapeutic aerosol mist either by means of acceleration of a compressed gas, typically air or oxygen, through a narrow venturi orifice or by means of ultrasonic agitation. Suitable formulations for use in nebulizers consist of the active ingredient in a liquid carrier, the active ingredient comprising up to 40% w/w of the formulation, but preferably less than 20% w/w. the carrier is typically water or a dilute aqueous alcoholic solution, preferably made isotonic with body fluids by the addition of, for example, sodium chloride.

As another example, a single dose of mersacidin can range from about 30-100 mg/kg, more typically about 50 mg/kg, applied in a volume of 2×10 μL to the nares of birds.

In one embodiment, mersacidin and/or the bacterial strains are administered as soon it is first suspected that the subject has a respiratory viral disease. Examples of early symptoms include respiratory symptoms, fever, cough, shortness of breath, breathing difficulties, muscle/joint pain and the loss of the sensation of taste/smell. The treatment can be continued for the duration of the disease. Treatment is considered effective if the disease is shortened in length or severity, or a symptom is decreased in length/severity. In one embodiment of the present invention, mersacidin and/or the bacterial strains are administered prophylactically (i.e., before exposure to a virus) to a subject who is at risk of developing a respiratory disease.

1. A method of treating a viral respiratory disease, comprising:
administering to a subject, in need thereof, an effective amount of L. reuteri strain 3632 and/or strain 3630, wherein L. reuteri strain 3632 and/or strain 3630 secrete mersacidin.

2. A method according to claim 1 wherein L. reuteri strain 3632 and/or strain 363 is administered by probiotic composition.

3. A method according to claim 1 wherein L. reuteri strain 3632 and/or strain 363 is administered by a live delivery platform.

4. The method according to claim 2 wherein the administration is oral, by inhalation, intravenous, subcutaneous, intramuscular or mucosal.

5. The method according to claim 1, wherein the disease is caused by a corona virus or influenza virus.

6. The method according to claim 5, wherein the corona virus is selected from the group consisting of alpha coronaviruses, the beta coronaviruses, MERS-COV, SAR-CoV, SARS-COV-2 and the common cold.

7. A method of treating a viral respiratory disease, comprising:
systemically administering to a subject, in need thereof, a pharmaceutical composition comprising an effective amount of mersacidin.

8. The method according to claim 7 wherein the administration is oral, by inhalation, intravenous, subcutaneous, intramuscular or mucosal.

9. The method according to claim 7, wherein the disease is caused by a corona virus or influenza virus.

10. A method of treating gut dysbiosis, comprising:
administering to a subject, in need thereof, an effective amount of L. reuteri strain 3632 and/or strain 3630, wherein L. reuteri strain 3632 and/or strain 3630 secrete mersacidin.

11. A method according to claim 10 wherein L. reuteri strain 3632 and/or strain 363 is administered by probiotic composition.

12. A method according to claim 10 wherein L. reuteri strain 3632 and/or strain 363 is administered by a live delivery platform.
